(19) United States
(12) Patent Application Publication
(10) Pub. No.: US 2009/0203067 A1
(43) Pub. Date: Aug. 13, 2009

(54) Photobioreactor Systems and Methods for Growing Organisms

(76) Inventors: Matthew W. Eckerle, Oakland, CA (US); Thomas W. Chalberg, JR., Redwood City, CA (US); Cheryl A. Hackworth, San Jose, CA (US); Michael B. Fertik, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 12/233,564

(22) Filed: Sep. 18, 2008

Related U.S. Application Data
(60) Provisional application No. 60/973,423, filed on Sep. 18, 2007.

(51) Int. Cl. C12P 1/00; C12M 1/00

(52) U.S. Cl. 435/41; 435/292.1; 435/288.7

(57) ABSTRACT

Photobioreactors and systems for growth of a photosynthetic organism are provided herein. The systems and photobioreactors can comprise features and modifications in order to improve photosynthetic growth efficiency and light energy utilization. Also provided are methods and systems to improve the cost-effectiveness of a photobioreactor system for growth of a photosynthetic organism.

### CROSS-REFERENCE

This application claims the benefit of U.S. Provisional Application Nos. 60/973,423 filed on Sep. 18, 2007, which application is incorporated herein by reference.

### BACKGROUND OF THE INVENTION

Mass cultivation of algae has been utilized for creating nutritional supplements, fertilizer, and food additives. In recent years, commercial growth of algae has also been explored to create biologically-derived energy products such as biodiesel, bioethanol, and hydrogen gas. When compared to terrestrial crops that can be used for biofuels, such as corn, soybeans, and sugarcane, algae can grow much faster and can produce up to 30 times more biomass per acre than the next most efficient crop (“A Look Back at the US Dept of Energy's Aquatic Species Program: Biodiesel from Algae.” NREL, 1998). Unlike terrestrial plants, which have roots and leaves, algae biomass is generally less specialized, and most or all cells can be used in conversion to fuel.

Many current methods of mass cultivation of algae involve open raceway pond systems, in which the algae is left open to the environment in a shallow, open air pond that is continuously stirred by a rotating arm or paddlewheel. These open raceway pond systems can provide a low cost growing environment, but can have some notable limitations including evaporation, temperature control, invasion of contaminating organisms, and light control. These limitations can reduce the productivity levels of these ponds, making them less suitable for their intended use and less cost-effective. Other approaches, including the use of closed-system bioreactors, have tried to overcome many of the issues associated with open ponds; these systems, however, can be less cost-effective than the open ponds. The design and demonstration of a method and system for the mass cultivation of algae that can address the limitations of the open raceway ponds has been challenging and elusive.

### SUMMARY OF THE INVENTION

The photobioreactors (PBRs) provided in accordance with the invention are closed systems for containing biomass that responds to light such as photosynthetic plants or algae. Such PBRs receive light as input to produce useful kinds of biological products. Different aspects of the invention provide optimal or improved growth rates which often rely upon incident light to fall within specific or desired intensity ranges. Furthermore, the invention provides solar photobioreactors and methods of their use that can change size and form in order to maintain internal light levels for photosynthesis. Preferable embodiments of the invention can also utilize or maximize available sunlight. Various components and apparatus can be combined with the photobioreactors in an integrated manner. The invention can facilitate the growth and harvest of organisms such as microalgae with efficient equipment that provides heating, cooling, and circulation of growth medium including apparatus driven by low-pressure hydraulics.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of many of the features and advantages of the invention will be obtained by reference to the following detailed description that sets forth illustrative embodiments, in which many of the principles of the invention are utilized, and the accompanying drawings of which:

FIG. 1 demonstrates an embodiment of the photo bioreactor of the invention comprising a means for mixing medium within the reactor, means for moving medium in and out of the reactor, and means for harvesting algae or biomass from the reactor. The photobioreactor is also capable of changing the depth within the photobioreactor.

FIG. 2 demonstrates the pumping action of a diaphragm in an example embodiment of the photobioreactor.

FIG. 3A demonstrates a photobioreactor having ribs which are oriented north-south for improving the direction of light entering the photobioreactor from various angles throughout the day.

FIG. 3B demonstrates an embodiment of the invention with a modified surface to insulate the contained biomass.

FIG. 4 demonstrates a means and method of controlling the temperature within the photobioreactor with air or gas.

FIG. 5 demonstrates a means and method of controlling the temperature within the photobioreactor with a liquid.

FIG. 6 demonstrates the effect of the angle of incidence from a light source on the amount of light reflected on the surface for different types of conditions.

FIG. 7A demonstrates a method of in which the orientation of the photobioreactor can be changed according to the position of the sun throughout the day in an attempt to minimize the angle of incidence of light energy to the surface of the photobioreactor.

FIG. 7B demonstrates a method in which the photobioreactor can be positioned to minimize the angle of incidence of light energy from the sun according to the season.

FIG. 8 illustrates a method of changing the orientation of a photobioreactor of the invention accomplished with a low-pressure hydraulic system.

FIG. 9 demonstrates an example control system for a photobioreactor of the invention.

### DETAILED DESCRIPTION OF THE INVENTION

Photobioreactors provided in accordance with the invention can be closed systems containing biomass that respond to light (for example, photosynthetic plants or algae). The photobioreactors can efficiently receive light as an input to produce biological products. In preferable embodiments of the invention, photobioreactors may comprise a light source that can provide controlled or improved growth relying upon incident light falling with specific or predetermined intensity ranges. Disclosed herein are also exemplary solar photobioreactors, preferably kept outdoors, that can change internal volume and/or form or shape to maintain internal light levels for photosynthesis within the photobioreactors. These and other solutions described herein can improve the utilization of available sunlight. The photobioreactors herein can be configured such that changes in internal volume or form, heating, cooling, and circulation can utilize low-pressure hydraulics and related apparatus.

An aspect of the invention provides photobioreactors that comprise: a reactor body formed with an inlet and an outlet that contains a liquid growth medium, wherein the liquid growth medium includes a photosynthetic organism; and a diaphragm within the body configured to move according to a set of instructions from a control system. For example, the body of the photobioreactor can be of any three dimensional shape as would be suitable to contain a liquid growth medium. A control system can comprise a sensor positioned on or near the body as described herein. A control system can also comprise a programmable processor or other computer control system as described herein. In an instance, a control system is a manual system as operated by a user. For example, a user may physically move the diaphragm. In another example, a user may provide input to a control system as described. In some instances, a control system operates automatically, for example, based on information detected by a sensor thereof, as described.

In another aspect of the invention, systems are provided for growing photosynthetic organisms that comprise: a plurality of photobioreactors, wherein each of the photobioreactors comprise a body formed with an inlet and an outlet, plus a liquid growth medium contained therein, wherein the liquid growth medium includes a photosynthetic organism; and a pump configured to move liquid growth medium through the inlet and the outlet of the more than one photobioreactor. The plurality of photobioreactors can be connected in series or in parallel or in series and in parallel as described herein. In some embodiments, at least one photobioreactor of the plurality of photobioreactors is configured to be removeably detached from the plurality of photobioreactors. Embodiments of photobioreactors as described herein can be part of a system for growing a photosynthetic organism that comprises a plurality of photobioreactors. For example, the exemplary photobioreactors as described may be individual photobioreactors in a photobioreactor system.

In some instances, the invention can address limitations of open-pond systems and other photobioreactors. The technology described herein has several features including, but not limited to: dynamic form which maximizes utilization of incident sunlight, modularity that affords efficient swapping of units, use of inexpensive materials, and low energy requirements. Closed systems, for example some of those described herein, can also limit problems associated with evaporation of water, allow for recycling of used water and medium, and reduce the chances of introducing foreign organisms or contamination.

The photobioreactors provided in accordance with the invention can allow for cost-effective cultivation of microalgae on a large scale. In an example, the photobioreactor is designed to provide more favorable and improved use of energy inputs (for example, light) for growing microalgae as compared to other photobioreactor devices and systems. Photosynthetic organisms such as algae use sunlight as an energy source to grow, divide, and/or make products, where the organisms and/or products can be used to create an eventual product output of the bioreactor. In some instances, too much sunlight can be detrimental to photosynthetic or algal growth, resulting in what is described as phototoxicity. Phototoxicity is caused by excessive light penetrating or impinging upon a cell and can lead to reduction in biosynthesis efficiency and, in some cases, can lead to cell death. Studies on phototoxicity indicate that light intensity should be regulated along with monitoring specific wavelengths of light for improvement of biosynthesis efficiency.

In a preferable embodiment of the invention, a photobioreactor can improve the light conditions for growing algae by providing the enhanced light intensity and wavelengths in which the algae will grow as compared to some other techniques. In some instances, the photobioreactor can also eliminate or reduce an over-abundance of light intensity, limiting phototoxic light levels within the bioreactor, and can block harmful wavelengths of light from entering the bioreactor.

Some of the photobioreactors herein may be described as apparatus containing, or configured to contain, a medium comprising at least one species of photosynthetic organism. As used herein, a liquid growth medium can describe a medium comprising at least one of water, nutrients for photosynthetic growth, and/or other liquid suitable for growing photosynthetic organism. In many instances a liquid growth medium comprises a photosynthetic organism, such as algae. As used herein, medium, growth medium, and liquid growth medium can, in many descriptions herein, be used interchangeably or in place of one another and certain vocabulary is used by way of example only. The photobioreactor can also have either a source of light capable of driving photosynthesis associated therewith, or have at least one surface at least a portion of which is partially transparent to light of a wavelength capable of driving photosynthesis (for example, light of a wavelength between about 400-700 nm). In some embodiments, energy (or light) from the sun is used to drive photosynthesis. Many of the photobioreactors described herein comprise a fully or partially enclosed bioreactor system, as contrasted with an open system, such as a pond or other open body of water, open tanks, and open channels.

A photosynthetic organism or biomass, as used herein, includes all organisms capable of photosynthetic growth, such as plant cells and microorganisms in unicellular or multi-cellular form and products produced by the photosynthetic organism. In most instances, a photobioreactor contains or is configured to contain a photosynthetic organism that is capable of growth in a liquid medium. Organisms suitable to be contained within a photobioreactor herein can include organisms modified by, for example without limitation, natural selection, selective breeding, directed evolution, synthetic assembly, or genetic manipulation. While many of the photobioreactors disclosed herein are particularly suited for the cultivation of algae, one skilled in the art can recognize that other photosynthetic organisms may be utilized in place of or in addition to algae. Many of the features, methods and embodiments of the photobioreactors described herein could be useful for other photobioreactor systems such as those described in U.S. Patent Application Publication 2005/0260553, U.S. Patent Application Publication 2007/0048859, and U.S. Patent Application Publication 2003/0228684, which are incorporated by reference herein in their entirety.

An example of biomass or a photosynthetic organism that can be grown within a photobioreactor of the invention is photosynthetic microalgae. Types of microalgae include, but are not limited to, cyanobacteria species (for example, microcystis), green algae (for example, Chlorella, Botryococcus, Ankistrodesmus, Chlamydomonas, Dunaliella), and diatoms (for example Thalassiosira, Navicula). The photobioreactor can also include heterotrophic growth as a component. In some instances, the algae are cultivated in a suitable medium that supports either autotrophic or both autotrophic and heterotrophic growth. A photobioreactor may also be utilized for the cultivation of multicellular aquatic plants, including macroalgae such as seaweeds and red algae.

In a preferable embodiment of the invention, closed system photobioreactors are provided which enable efficient levels of algal autotrophic growth (for example, using only inorganic carbon as a carbon source). The photobioreactors can also utilize conditions using autotrophic and heterotrophic growth in combination (for example, using carbon dioxide and other organic molecules, such as sugar or starch, as the carbon source), also known as mixotrophic growth.

Algal cells can be kept at a density within a photobioreactor whereby cells divide at a logarithmic rate. To maintain a desired cell density in the culture with growing and dividing cells, fresh medium (for example, water, water with sugar, and plant food) may be directed into the photobioreactor to dilute the culture, and algae and medium may be directed out of the photobioreactor to be harvested. In order to be harvested, medium containing algal biomass can be pumped out of the bioreactor, or can flow out or spillover as a consequence of fresh medium being added.

The medium that can be directed into the photobioreactor can comprise water or a saline solution (for example, sea water or brackish water) containing sufficient nutrients to facilitate viability and growth of algae and/or other photosynthetic organisms. In an embodiment, a medium can be utilized that comprises brackish water, sea water (filtered or unfiltered), or other non-potable water. In other embodiments, a medium comprises waste water, such as grey water, water from storm drains, or water from sewage treatment plants. The medium can be obtained from a locality in which the photobioreactor will be operated. Particular medium compositions containing water plus nutrients required or suitable for use in maintaining a growing culture of algae or other photosynthetic organism are well known in the art. Potentially, a wide variety of media can be utilized in various forms for various embodiments of the invention, as would be understood by those of ordinary skill in the art. In many embodiments of the invention, the medium includes a source of nitrogen, phosphate, and micronutrients such as essential metals. Examples of media include, but are not limited to, High Density Growth of Chlorella medium, Biogenesis, Ironite, F2, Proteose, Alga-Gro, and media containing Hoagland's salt mixture. Media may additionally contain a protein or sugar source.

Another embodiment of the invention provides a photobioreactor that has variable depth ranges to control the average light intensity exposure. The depth can be controlled to respond to one or more of the following variables: the amount of solar radiation (sunlight) available to the photobioreactor as a function of time of day, season, weather, location, or other shading; the amount of medium or biological material available, or present within the photobioreactor; and the type of medium or biological material inside the photobioreactor. A series of hydraulic pumps and related apparatus described elsewhere herein may regulate or modify the desired variable depths of the photobioreactors.

An optimal depth of the photobioreactor may be described as where the average position of a cell inside the photobioreactor is exposed to the minimum light intensity required for the maximal photosynthetic growth (described as approximately 250 foot-candles in Sorokin and Krauss “Effects of Temperature & Illuminance on Chlorella Growth Uncoupled From Cell Division.” Plant Physiol., 1962; 37(1):37-42). When more light is provided at the same depth, no further growth is achieved; an abundance of light can cause slower growth due to photoinhibition or phototoxicity.

The average internal light in a photobioreactor is a function of incident light, surface light absorption efficiency, light absorption per unit depth (optical density), and overall depth. For a given density of algal cells suspended in medium, the light intensity at a given depth is determined by Beer's law shown in Equation (1),

I(x)=I(0)*e^{-(x*alpha)} (1)

where I(0) the incident light intensity at depth zero, x is the actual depth and alpha is the actual absorption coefficient. The average internal light level can therefore be calculated.

If a medium is mixed or stirred within a photobioreactor, a given algal cell will experience a distribution of light intensity, and for a given surface intensity, a target average intensity can be controlled based on the depth of the photobioreactor. This depth can be set to maximize or improve the light utilization as described above.

A medium can be “self-shading”, where the cells near the bottom are shaded by the cells above. In a dense medium, cells more than a few centimeters below the surface may receive very little light. If the medium is mixed rapidly, this could produce the “flashing light effect”, which shows that algae can grow as efficiently with intermittent exposure to light as under constant light at the same intensity. (“A Look Back at the US Dept of Energy's Aquatic Species Program: Biodiesel from Algae.” NREL, 1998, and references therein.)

In an embodiment of the invention, a photobioreactor comprises a sensor configured to detect light intensity at a surface of a body of a photobioreactor. Surface light intensity can vary according to, for example: time of day (for example, more incident light intensity at noon); time of year (for example, more incident light intensity in summer); and weather (for example, more incident light intensity without clouds, greenhouse gases).

Surface illuminance can be detected at a site comprising photobioreactors, for example, in a field comprising a plurality of photobioreactors. Illuminance can be measured centrally or in a grid around the site for increased resolution. Illuminance may be measured over a broad spectrum of light or at a specific wavelength (for example, 450 nm) to measure the primary wavelengths involved in photosynthesis. The surface illuminance can be measured on a photobioreactor. Any of several known methods can be used to detect illuminance, for example a photodiode could be used. Other examples of types of sensors for measuring surface illuminance include, but are not limited to, fluorescent pigments, quantum dots, photoresistors, and thermal black body (bolometer-type). Alternatively, surface illuminance can be approximated by satellite imagery. (“Validation of GOES-based insolation estimates using pyranometer insolation data from the United States Climate Reference Network.” 26th Conference on Agricultural and Forest Meteorology, 2004.)

In some embodiments of the invention, one or more sensors are configured to detect the optical density of the photosynthetic biomass medium provided within a photobioreactor. Optical density of the culture can be measured by comparing surface light intensity measurements with measurements made at a known depth in the photobioreactor using methods and systems described herein. Alternatively, optical density can be measured downstream from the photobioreactor in the medium containing algae that is being harvested. The optical density of the culture can also be detected by a sensor. Examples of sensors for detecting optical density include, but are not limited to, a turbidity meter and spectrophotometer.

Depending on the surface illuminance, the depth of the culture can be varied to maintain the target average light intensity. Incident light and optical density of the culture can be used to calculate the optimal depth relative to the light-absorbing surface(s) of the bioreactor for optimal light levels for photosynthesis at any given time.

In another embodiment of the invention, a photobioreactor is provided that comprises: a body comprising an inlet and an outlet and containing a liquid growth medium, wherein the liquid growth medium comprises a photosynthetic organism; a diaphragm within the body configured to move according to a set of instructions from a control system; and a diaphragm pump in communication with the diaphragm. In some instances, the diaphragm pump is a hydraulic pump configured to move a medium into the diaphragm according to the set of instructions from the control system.

The change in form required to create variable depth of the biomass being grown within the reactor may be driven by low-pressure hydraulics. Fluid in flexible chambers of the bioreactor can form a low-pressure, shape-changing hydraulic circuit that raises or lowers a diaphragm to change the bioreactor depth. The movement of the diaphragm to change the depth can improve internal illuminance while using very low power. For example, a photobioreactor can comprise a body configured to contain an algal medium and a diaphragm, wherein the diaphragm is movable in order to change the depth of the algal medium relative to an illuminated surface or an illumination source. In some instances, the depth is the distance between algal biomass cells and the light source or illuminated surface. Alternatively, the depth can be a distance between the diaphragm and an exterior surface of the photobioreactor that is transparent to light. In some instances, biomass is harvested from a photobioreactor when the diaphragm changes the volume of the photobioreactor available to a medium.

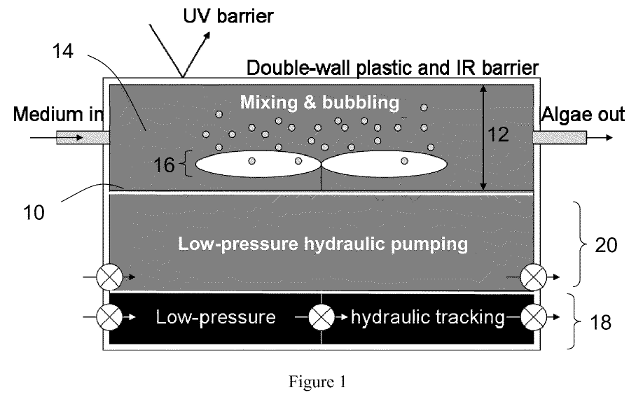

FIG. 1 demonstrates an exemplary embodiment of a photobioreactor of the invention capable of moving a diaphragm (10) up or down, thereby contracting or expanding, respectively, the depth (12) and/or interior volume (14) of the photobioreactor. The interior volume (14) of the photobioreactor can be configured to contain a growth medium. A photobioreactor can also be capable of tracking or altering its orientation, for example by utilizing a programmable stage (18) as explained herein. The photobioreactor in FIG. 1 also comprises a mixer for mixing the medium within the reactor, and an inlet and outlet for moving medium in and out of the reactor as necessary, wherein the outlet can be used to harvest algae or biomass from the reactor, as described in detail herein.

The pumping of liquid medium and gas in and out of a photobioreactor, the size and form modification of the photobioreactor, the circulation of the biomass inside the photobioreactor, and the harvesting of biomass from the photobioreactor can all or in part be driven by low pressure hydraulics. For example, low pressure hydraulics can be water pressure produced by an elevated water source (for example, a water tower), so as to minimize energy inputs required.

The low-pressure hydraulic system can comprise a diaphragm pump (20) as demonstrated in FIG. 1. A diaphragm pump is a positive displacement pump that uses a combination of the reciprocating action of a flexible diaphragm and suitable non-return check valves to pump a fluid. Sometimes this type of pump is also called membrane pump. If the biomass is grown in contact or a medium is in contact with the diaphragm pump, the reciprocating action of the pump can be controlled to vary the depth of the biomass within the photobioreactor. For example, in the embodiment demonstrated in FIG. 1, valves are used to control the volume within the diaphragm. Biomass growing on or near the diaphragm becomes closer to the surface of the photobioreactor where the light energy enters when the volume of the diaphragm chamber is greater. The biomass can also be grown in the medium and mixed by a mixer within the photobioreactor of FIG. 1 for example as shown by the fan and bubbles. When mixed, the population of algae cells can experience an average light intensity. Depending on the volume in the diaphragm pump as controlled by the valves and/or a control system, the algae can have a greater or lesser volume to occupy within the photobioreactor.

Diaphragm pumps have a diaphragm through which repeated compression/decompression motion is transmitted. In many instances, the liquid does not penetrate through the diaphragm, so the liquid inside the pump is sealed off from the outside. Such motion changes the volume of a chamber so that liquid enters through an inlet check valve during decompression and exits through an outlet check valve during compression. The diaphragm can be flexible, and can be fixed inside the photobioreactor via a fitting or, alternatively, be part of the bioreactor (for example, via welding). The diaphragm may be uniform in thickness, or may vary somewhat in thickness or shape, as the processes may require.

Diaphragm metering pumps or diaphragm pumps are commonly hydraulically driven. Hydraulic pumps are positive displacement pumps, meaning that the flow is directly related to the displacement of the pump and its speed. Diaphragm pumps have flow rates that are often dependent on the effective working diameter of the diaphragm and its stroke length. They can handle sludges and slurries with a good amount of grit and solid content. In some instances, diaphragms or diaphragm pumps can move medium comprising a dense population of cells, such as algal cells.

Types of diaphragms include rubber, plastic (clear or colored), air-permeable membranes, and composite materials. The pumps can also include ball check valves and actuators to deliver precise volumes. Types of actuators include, but are not limited to, hydraulic, pneumatic, electric, and thermal actuators.

The diaphragm can also act to pull medium and/or biomass into and push medium and/or biomass out of the photobioreactor. When the volume of a chamber of a diaphragm is decreased (the diaphragm moving down), the pressure decreases, and fluid can be drawn into the chamber. When the chamber pressure later increases from increased volume (the diaphragm moving up), fluid can be forced out.

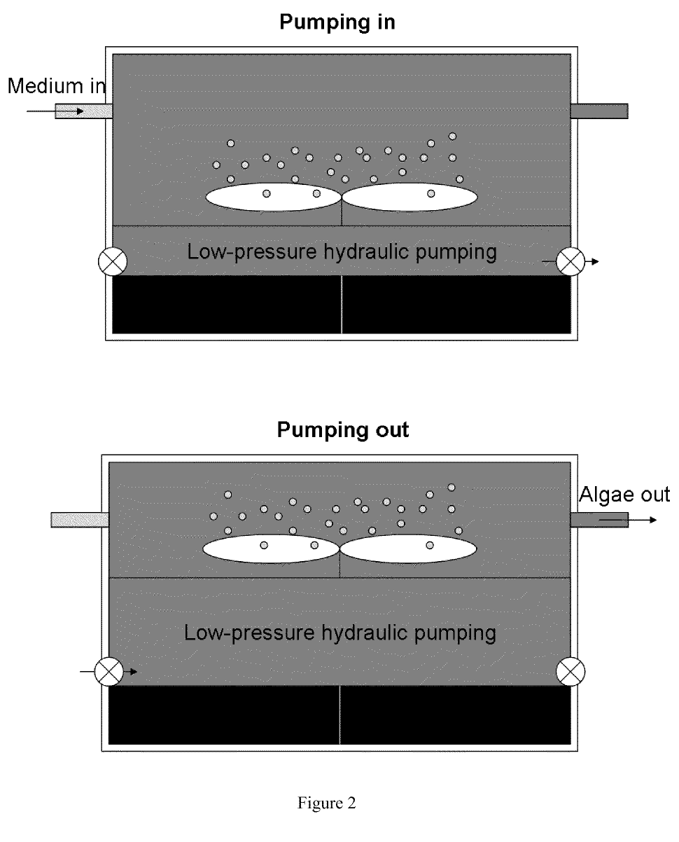

FIG. 2 demonstrates the pumping action of a diaphragm in an exemplary embodiment of the photobioreactor. As the volume in the diaphragm decreases, medium can be drawn into the photobioreactor, and as the volume in the diaphragm increases, medium and/or algae can be outputted from the photobioreactor. The interior volume within the photobioreactor for containing medium can be reduced from its maximal interior volume by at least about 2, 5, 10, 15, 20, 25, 30, 35, 40, 45, 50, 60, 70, 80, 90, 95, or 97 percent by movement of the diaphragm. In a similar manner, the medium can be continuously cycled to improve growth conditions, or can be controlled in terms of medium volume and time of medium distribution to the photobioreactor.

The diaphragm pump may be driven by water pressure or air pressure generated external to the bioreactor by pumping or elevation of fluid. Other types of hydraulic pumps or motion devices that can be incorporated into a photobioreactor of the invention include, but are not limited to, screw pumps, gear pumps, geroter pumps, vanepumps, axial piston pumps, radial piston pumps, and motors including axial piston, diaphragm, gear, vane, screw, and geroter motors. A single or a plurality of hydraulic and pneumatic motion devices may be integrated into this process to realize the features of different photobioreactors of the invention.

The photobioreactor surface(s) of light absorption may be physically or chemically modified to improve light absorption and/or a chosen spectral component. This can include modification to reflect undesired light (for example, ultraviolet light) while absorbing desired light (for example, photosynthetically active radiation (PAR) between 400 and 700 nm or light in the visible spectrum) from any angle of incidence.

In some instances, a body of a photobioreactor comprises a wavelength selective surface configured to allow selected wavelengths of light through the surface and block unselected wavelengths of light. For example, the selected wavelengths can be predetermined or preselected by a user or manufacturer of a bioreactor or system as described herein. In many instances, the selected wavelengths are suitable wavelengths for providing energy for photosynthesis to an organism to be grown within the bioreactor. For example, in some cases, the selected wavelengths may be wavelengths for growing a specific species of algae in which that wavelength improves the growth of the algae and can be different for different species of algae. In some embodiments, the selected wavelengths are in the visible spectrum.

In other instances, unselected wavelengths can be those not suitable for photosynthetic growth of an organism contained within the body of the bioreactor. For example, a material can be utilized that blocks UV or IR light from entering the photobioreactor, but allows visible light to pass through to the biomass contained within the reactor. In some embodiments, the unselected wavelengths are in the UV spectrum.

In some embodiments, a wavelength selective surface comprises a wavelength selective coating. In some embodiments, a wavelength selective surface comprises a wavelength selective plastic.

A wavelength selective surface can involve adding chemicals to the plastic (for example, doping the plastic), layering different kinds of plastic, coating plastic with other chemicals. For example, in FIGS. 1 and 2, the photobioreactors have a surface that is penetrable by the desired light wavelengths. In the examples of the figures, a wavelength selective surface can be a double-wall wavelength selective plastic that is capable of being a barrier to both the UV and IR spectra of light energy, while allowing light energy in the visible spectrum through to the biomass growing within the photobioreactor.

Examples of types of wavelength selective coatings for a photobioreactor include, but are not limited to, coatings that block ultraviolet light, coatings that prevent light-induced degradation of the plastic, coatings that block IR light, coatings that minimize reflectance at certain wavelengths, coatings that maximize reflectance at certain wavelengths, coatings that minimize/maximize transmission of certain wavelengths, and coatings that minimize/maximize absorption of certain wavelengths. A thin solar film coating could be added to capture light energy for conversion to electricity.

Since different types of biomass can require different light exposure conditions for optimal growth and proliferation, in some embodiments, especially those where sensitive algal species are employed, light modification apparatus or devices can be utilized in the construction of a photobioreactor. For example, some species either grow much more slowly or die when exposed to ultraviolet light (Reference: “Monochromatic Light Saturation Curves for Photosynthesis in Chlorella”. J M Pickett and J. Myers. Plant Physiology. 1966. 41: 90-98). If the specific algae species being utilized in the photobioreactor is sensitive to ultraviolet light, then, some portions of external surface of photobioreactor could be covered with one or more light filters or coatings that can reduce transmission of the undesired radiation. Such a light filter can permit entry into the photobioreactor of wavelengths of the light spectrum that the algae need for growth while barring or reducing entry of the harmful portions of the light spectrum. Such optical filter technology is already commercially available for other purposes (for example, for coatings on car and home windows) that may be useful with embodiments described herein. A wide variety of other optical filters and light blocking/filtering mechanisms suitable for use in the above context will be readily apparent to those of ordinary skill in the art.

The photobioreactor can be made of plastic. Different types of plastic may be used for their desirable properties on the different bioreactor components or be laminated to each other to combine their properties. For example, rigid PVC may be used for components requiring stiffness and exceptional strength while plasticized PVC may be used for components requiring flexibility and light transmittance, or polyethylene and nylon may be laminated to join polyethylene's vapor barrier with nylon's strength. The plastic may be treated for resistance to UV radiation to prevent degradation of the plastic and to decrease photoinhibition of the algal growth. The photobioreactor may be made of materials that support the cost-effective production of low-value products (such as fuel) as well as high-value products (such as cosmetics or nutritional supplements). The photobioreactor can be made of other materials, such as metal, glass, concrete, and rubber. The photobioreactor or components of the photobioreactor can be made of recycled materials (for example, recycled plastics).

In many instances, in order to grow biomass within a photobioreactor, gas is pumped into the bioreactor. The gas used can be air, filtered air, or unfiltered air that may or may not be supplemented with certain gases (for example, carbon dioxide) to improve growth and algal productivity. The gas can also be waste gas, such as flue gas from a coal or natural gas fired power plant. Gas (for example, oxygen) can also be removed from the medium. The gas entering the photobioreactor can also be used for heating or cooling as well as mixing as described herein.

The biomass and medium inside the reactor may be mixed. In some instances, mixing can allow for dispersal of nutrients and carbon dioxide, efficient removal of waste products, maintenance of optimal light conditions for each individual algal cell, and reduction of algal colony adherence on the walls of the bioreactor. Mixing can be achieved through the pumping of water and/or medium into the bioreactor. It can also be achieved using by bubbling gas into the bioreactor, or by using pneumatics. In an exemplary embodiment as demonstrated in FIGS. 1 and 2, the bioreactor may contain a fan or propeller for mixing that can move about a pivot axis and circulate medium, and gas within the bioreactor. This propeller can be turned by using a stream of water (hydraulics) or air (pneumatics) to achieve mixing. The propeller can also be driven by any other type of motor as would be evident to those skilled in the art. The mixing can also occur through the motion of the diaphragm. In an embodiment comprising a diaphragm, the hydraulics can be the same or different than the system for variable depth control or pumping.

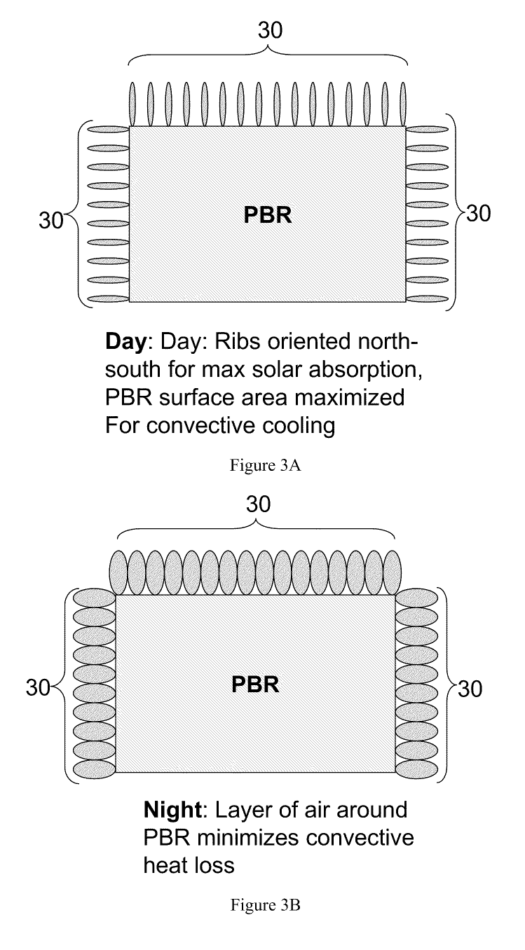

In preferable embodiments of the invention, the surface of the bioreactor contains a special texture to improve the direction of light into the bioreactor. The direction of light entering the photobioreactor can be improved by having ribs (30) which are oriented parallel to the direction that the sun travels, as demonstrated in FIG. 3A. For example, the ribs can be oriented in a north-south direction during the day.

In various embodiments of the invention herein, a photobioreactor can be designed to provide temperature control in order to improve growth of a biomass. The form of the surface of the bioreactor may be changed for cooling during the day and heat retention at night. For example, the surface of the bioreactor may be modified to insulate the contained biomass, as demonstrated in FIG. 3B. The ribs can be moved to a position such that convective heat transport to or from the photobioreactor is reduced or such that a layer of air is trapped around the photobioreactor.

The heating needs of the bioreactor may be drastically reduced by insulation, which can be economically achieved by employing a layer of air trapped in plastic surrounding the bioreactor (double-paned plastic) to reduce convective heat loss. Heating needs can be further reduced by modifying the bioreactor surface to reflect infrared light, thus keeping it from escaping and preventing irradiative heat loss.

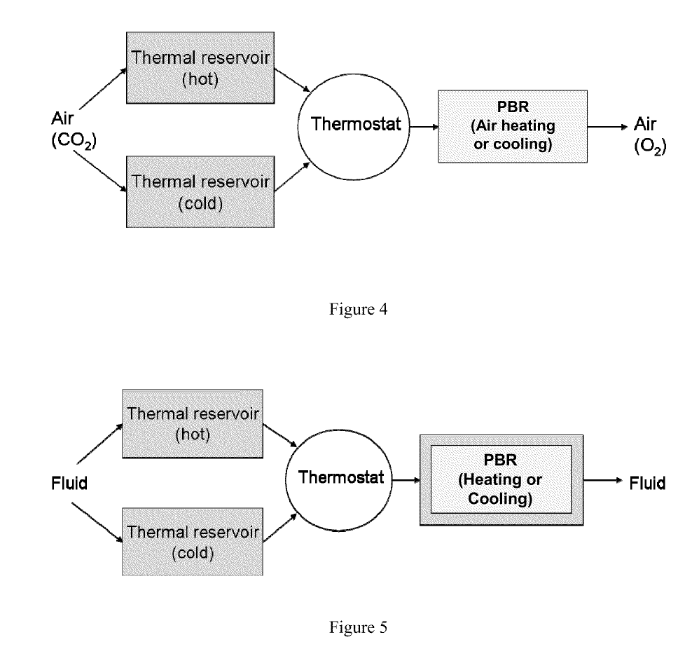

FIG. 4 demonstrates a means and method of controlling the temperature within the photobioreactor. Air or gas (for example, air or carbon dioxide) can travel through or be contained within a thermal reservoir at a high (or hot) temperature that is above the desired culture temperature (for example, 35-40° C. where the desired growth temperature is ˜27° C.). A separate amount of air can travel through or be contained within a thermal reservoir at a cooler (or cold) temperature that is below an optimal growth temperature (for example, 10° C. where the desired growth temperature is 27° C.). Depending on the desired temperature within a photobioreactor, a sensor, such as a thermostat, can control the temperature of the gas delivered to the photobioreactor, which in turn controls the temperature within the reactor. If a gas containing carbon dioxide is provided, as in the example of FIG. 4, the carbon dioxide can be used both to feed the biomass and control the temperature of the system. In some instances, gas expelled from the photosynthetic organism (for example, oxygen) can exit the photobioreactor and may or may not be used for other processes. Alternatively, gas can be circulated in a layer outside of the chamber containing biomass to heat or cool the biomass by heat transfer. The gas can be contained or circulate between layers of a photobioreactor comprising a double-wall plastic at the surfaces and edges. Gas in this layer can also be used to support the shape and structure of the bioreactor.

Another method and system of controlling the temperature of a photobioreactor is illustrated in FIG. 5. In this example, fluid can travel through or be contained within a thermal reservoir at a high (or hot) temperature. A separate fluid can travel through or be contained within a thermal reservoir at a cooler (or cold) temperature. Depending on the desired temperature within a photobioreactor, a sensor such as a thermostat, as shown in FIG. 5 can control the temperature of the fluid, and is then used to heat or cool the surfaces of the photobioreactor, which in turn controls the temperature within the reactor. A fluid used to control the temperature can be growth medium or water. The fluid can be mixed in direct contact with the medium inside the bioreactor, or alternatively, can be circulated in the space surrounding the bioreactor so as to transfer the temperature to the circulating biomass inside. This fluid can be contained or circulate between layers of a photobioreactor comprising a double-wall plastic at the surfaces and edges. When fluid is within the double-wall plastic it can be used to support the shape and structure of the bioreactor. Reservoirs, when used for controlling the temperature, can be simple or complex apparatuses. In some embodiments, the reservoirs are maintained by the temperature at which they are in relation to the surface of the Earth. For example, a reservoir 10 m underground might have a constant temperature of 13° C., and reservoirs at different underground depths can be used to store fluids at different temperatures. In one embodiment, fluid reservoirs are used in combination with heat pumps or air conditioners to create a geothermal system or a heating and cooling system. In other embodiments, the hot reservoir may be composed of solar energy absorbing material, whereas as the cold reservoir may be composed of a solar energy reflecting material. In another embodiment, the hot reservoir can comprise a heat conducting material exposed to higher temperature and the cold reservoir can comprise a material with poor heat conduction. In another example, fluid or gas can enter the hot reservoir or cold reservoir at a desired temperature, and the reservoirs can be insulated.

In a preferable embodiment, the orientation of the sun towards a photobioreactor can be altered or changed. When light strikes the surface of the photobioreactor at an angle, a portion of the light will be reflected back toward the atmosphere. In order to attempt to maximize capture of incident sunlight, the angle of incidence of solar radiation to the photobioreactor can be variable. For example, for a given position on the earth's surface, the angle of incident rays from the sun is variable according to: time of day (for example, East in AM, West in PM); time of year (for example, in Northern Hemisphere, it is south in summer and farther south in winter); and location (for example, latitude, longitude, elevation).

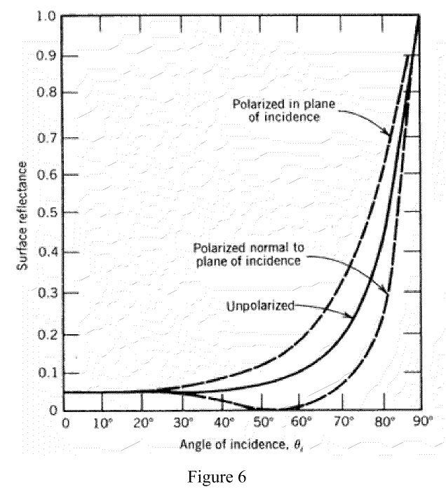

FIG. 6 demonstrates the effect of the angle of incidence from a light source on the amount of light reflected on the surface for different types of conditions. Light is minimally reflected and maximally transmitted when the angle of incidence is 0° relative to the planar surface of the material as shown in FIG. 6. Therefore in order to increase the amount of light inside the bioreactor, the surface can be tilted toward the sun, such that incident rays are normal to the planar surface of the bioreactor. Conversely, if it is desired to decrease the amount of available light (for example, to reduce phototoxicity), the planar surface can be tilted away from the sun.

Another aspect of the invention provides systems for growing a photosynthetic organism that comprises: a photobioreactor comprising a body comprising an inlet and an outlet and containing a liquid growth medium, wherein the liquid growth medium comprises a photosynthetic organism; and a programmable stage configured to couple to the photobioreactor, wherein the programmable stage is configured to move according to a set of instructions. In some instances, the set of instructions are from a user. The system can be orientated to reduce the angle of incidence of light from a light source, such as the sun. In an embodiment, a system further comprises a sensor configured to detect light intensity at a surface of the body. The set of instructions can be from the sensor or utilize information obtained from the sensor. In some instances, the set of instructions relate to the position of the sun in the sky throughout a day.

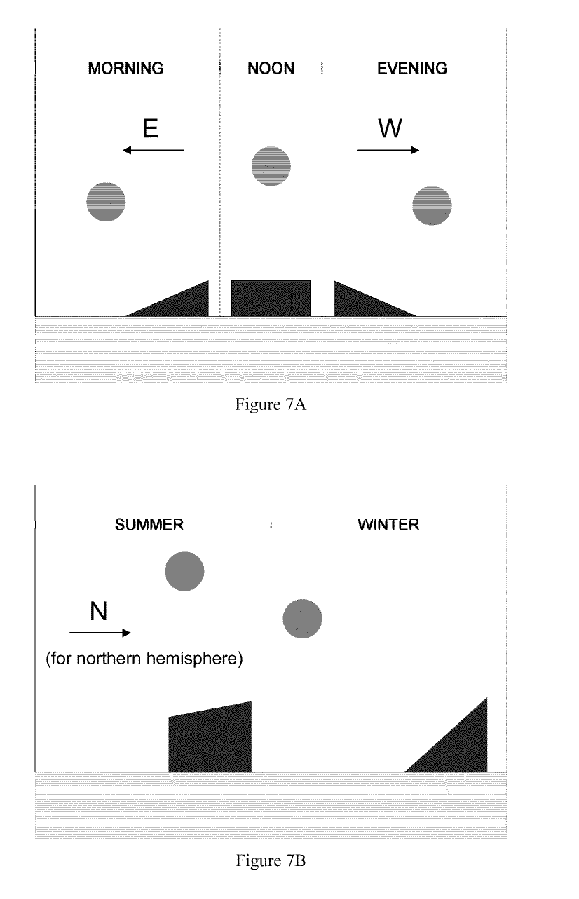

A photobioreactor or photobioreactor system can be oriented, angled or tilted toward the sun. The optimal east-west and north-south angles can be calculated with a solar position calculator for any point in time based on the known latitude, longitude and elevation of the photobioreactor. This orientation of the photobioreactor can be determined based on facing the photobioreactor in an improved manner for receiving light absorption by orienting the light-absorbing surface(s) normal to the sun at any time during the day/season. FIG. 7A demonstrates how the orientation of the photobioreactor can be changed according to the position of the sun throughout the day in an attempt to minimize the angle of incidence of light energy to the surface of the photobioreactor. The photobioreactor can also be positioned to minimize the angle of incidence of light energy from the sun according to seasons of a year as shown in FIG. 7B.

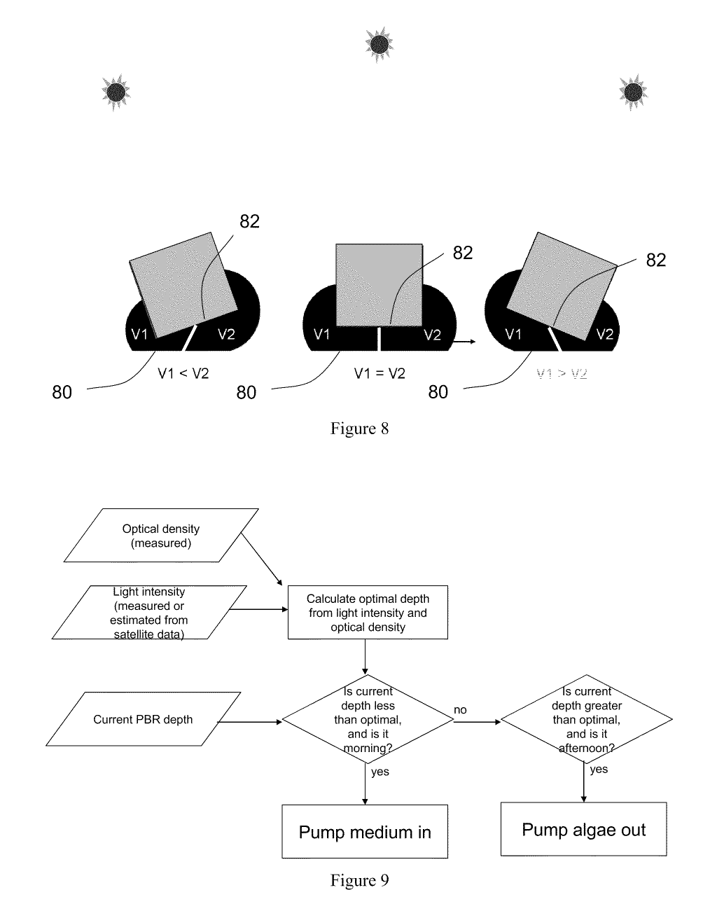

Changing the orientation of a photobioreactor or a series or field of photobioreactors can be accomplished using a low-pressure hydraulic system, such as the one described herein. For example, a programmable stage can have a low-pressure hydraulic system configured to be programmable according to a set of instructions. A programmable stage can also have a flat surface or any surface that is configured to move according to a set of instructions and couple to a photobioreactor or a plurality of photobioreactors as described herein. For example, FIG. 8 demonstrates two low pressure hydraulic diaphragms on which a photobioreactor rests or is coupled to. The photobioreactor can be positioned according to the amount of volume within each diaphragm. The photobioreactor can tilt at least about 2, 5, 10, 20, 30, 40, 50, 60, 70, 80, or 90 degrees. An angle of tilt can be measured between a first line normal to the surface (80) beneath the photobioreactor and a second line normal to a base (82) of the photobioreactor. The photobioreactor can also be positioned according to the movement of any sort of chamber capable of changing shape and/or size. For example, when one diaphragm has less volume than the other, the photobioreactor tilts in the direction of the diaphragm with less volume. When the volumes are equal, the photobioreactor can position itself straight up and down. Desired angles can be calculated based on the solar position, and can be achieved by varying the volume of each diaphragm. The volume in the diaphragm can be altered by pumping a fluid in and/or out by a low pressure hydraulic system, based on feedback or commands from a control system. The use of two diaphragms in FIG. 8 serves only as an example. Any number of diaphragms can be used based on the positioning of the photobioreactor and/or complexity of a system of photobioreactors. For example, four diaphragms can be used to obtain any orientation along two axes (for example, North-South and East-West). Such a low-pressure hydraulic system can be utilized alongside a variable-depth system also powered by low-pressure hydraulics.

The low pressure hydraulic system for tilting a photobioreactor can be configured in such a way that fluid within a first diaphragm can be transferred to a second diaphragm during tilting of the photobioreactor. This configuration can conserve the fluid medium contained within the diaphragms, reduce the need for additional reservoirs for storage of the fluid medium, and/or reduce the amount of energy required to tilt the photobioreactor.

Although the photobioreactors described herein are being described as utilizing natural sunlight, in alternative embodiments, an artificial light source providing light at a wavelength able to drive photosynthesis may be utilized instead of or in supplement to natural sunlight. Examples of artificial light sources include, but are not limited to, LEDs, light bulbs, halogen lamps, fluorescent paint, and the like. For example, a photobioreactor utilizing both sunlight and an artificial light source may be configured to utilize sunlight during the daylight hours and artificial light in the night hours, so as to increase the total amount of time during the day in which the photobioreactor can convert carbon dioxide to biomass through photosynthesis.

To reduce production costs, a photobioreactor as described herein can be made of inexpensive materials. A photobioreactor can be made of inexpensive thin plastic coated in a film that can maintain the life of the plastic while protecting the contained photosynthetic organisms. For example, the material chosen may limit the input of labor costs as compared to other known closed systems because many of the components, such as harvesting, mixing of the medium, and stirring the culture, can be automated with energy provided by low pressure hydraulics.

Control of the concentration of biomass within the photobioreactor can be important both from the standpoint of maintaining a desirable level of algal growth and proliferation. Biomass can be harvested periodically or continuously to maintain the desired concentration range during operation. In an embodiment, harvesting takes place in a continuous or semi-continuous fashion, meaning that biomass is constantly removed, or only a portion of the biomass is removed from the photobioreactor at a given time. Harvesting can be accomplished by pumping out medium, or by pumping in medium to displace biomass out of the bioreactor in a controlled way. If reducing the biomass volume is desired, the diaphragm may be used to push the biomass out of the photobioreactor. Harvested algae and medium can be separated, and medium can be tested and adjusted for re-use (for example, for nutrient concentration and pH). Medium may be filtered before use to ensure it is free of contaminants when entering the photobioreactor.

Water-rich air coming out of the photobioreactor may be put through a water condensing or water sequestering apparatus to decrease water loss from the system. Rainwater can be captured to make up for water loss or to expand the algal culture.

A plurality of photobioreactors can be arranged to from a system for the growth and production of a photosynthetic biomass. As would be apparent to those skilled in the art, in some embodiments, a photobioreactor system can comprise one of a plurality of identical or similar photobioreactors interconnected in parallel, in series, or in a combination of parallel and series configurations. For example, this could increase the capacity of the system (for example, for a parallel configuration of multiple photobioreactors). All such configurations and arrangements of the inventive photobioreactor apparatus provided herein are within the scope of the invention.

In some instances, each unit of a system of photobioreactors can operate independently. The units can be modular and they can be easily swapped if desired. For example, if one unit becomes contaminated with another species of algae or other organism, it can be swapped for a different unit.

Although a system of photobioreactors of the invention can be intended to be modular and self-contained, harvest processes, medium recycling, water storage, power generation, and other processes may be centralized and distributed to individual photobioreactors. Independent units are connected in a network so that dispersal of medium and collection of biomass products can be centrally coordinated.

In some embodiments a control system and methodology is utilized in the operation of a photobioreactor, which is configured to enable automatic, real-time optimization and/or adjustment of operating parameters to achieve desired or optimal photomodulation and/or growth rates for a particular environmental operating conditions. In yet another aspect, methods and systems are provided for preselecting, adapting, and conditioning one or more species of photosynthetic organisms to specific environmental and/or operating conditions to which the photosynthetic organisms will subsequently be exposed during utilization in a photobioreactor apparatus of a gas treatment system.

A computer implemented system can be used to control light exposure, media flow rates, gas exchange rates, orientation of a photobioreactor in respect to the sun, heating and cooling of the photobioreactor, mixing, low pressure hydraulic systems, and harvesting of the biomass. The computer control system can have the ability to adjust different parameters in an attempt to optimize log-based growth of the biomass in a photobioreactor of the invention. The system can be implemented to adjust parameters automatically. For example, a computer implemented system can calculate light exposure intervals to determine the duration of exposure of the biomass, on average, to light intensities both above and below an optimum intensity required to drive photosynthesis in a log-based manner. In another example, the system can determine the frequency of exposure of the algae to light and dark periods of the biomass.

FIG. 9 demonstrates an example control system for a photobioreactor of the invention. The light intensity at the surface of the photobioreactor can be measured by a sensor on or near the reactor, or estimated from satellite data. The optical density can also be measured by sensor on or near the reactor, or estimated from data at a remote location. From the measurements of light intensity and optical density, the optimal positioning or depth of the biomass culture and/or algae within the photobioreactor can be calculated using the methods, devices, and systems described herein. Taking into account the current position of the biomass culture within the photobioreactor and the optimal or anticipated position, the system can determine if it needs to move the position of the biomass. This is demonstrated by way of example in FIG. 9, wherein the system asks a question pertaining to the current depth versus the optimal depth and asks the time of day.

If the current depth needs to be adjusted, a photobioreactor of the invention can adjust the position of the biomass accordingly using a control system. To do so, the system can pump medium into the system or pump medium and algae out of the system, as shown in FIG. 9. In some instances, at least one photobioreactor of a plurality of photobioreactors further comprises a diaphragm within the body configured to move according to a set of instructions from a control system. The at least one photobioreactor of the plurality of photobioreactors can also further comprise a diaphragm pump in communication with the diaphragm. For example, as described herein, a diaphragm pump can be a hydraulic pump configured to move a fluid into the diaphragm according to the set of instructions from the control system. In some instances, a control system for a system plurality of photobioreactors comprises one sensor or a plurality of sensors positioned on or near the body of the photobioreactors, wherein the sensor is configured to detect light intensity at a surface of the body.

In embodiments utilizing a hydraulic diaphragm system, the pumping can be accomplished by pumping the diaphragm up or down. The diaphragm system can be controlled by a computer implemented method.

Control of the photobioreactor can be achieved using conventional hardware or software-implemented computer and/or electronic control systems together with a variety of electronic sensors.

In some instances, a system further comprises a temperature control system comprising a temperature sensor, a heater, and a cooler, wherein the temperature sensor is in contact with the liquid growth medium. For example, the temperature control system can be configured to adjust the temperature of a medium entering the inlet according to the temperature sensor.

For example, it can be important to control temperature within photobioreactor during operation to maintain the temperature of the medium and/or biomass at a range to improve productivity. Desirable temperature ranges for operation depend upon the characteristics of the biomass or photosynthetic organism grown in the photobioreactor. It can be desirable to maintain the temperature between about 5° C. and about 45° C. In an embodiment, the temperature is between about 15° C. and about 37° C. In a further embodiment, the temperature is between about 20° C. and about 30° C., or about 27° C.

Using the control systems of the invention, components for nutrient level maintenance, pH control, and other factors could be added automatically directly into the liquid phase within the photobioreactor, if desired. The control system can also be configured to control the temperature in the photobioreactor by either or both of controlling a heat exchanger system or heat control system within or connected with the photobioreactor.

In some embodiments, a system as described herein comprises a plurality of programmable stages configured to couple to the plurality of photobioreactors, wherein the programmable stages are configured to move according to a set of instructions, for example, to manipulate the position of the plurality of photobioreactors according to the angle of incidence originating from the light source as described. In some instances, the set of instructions are from a sensor configured to detect surface illuminance. The set of instructions can relate to the position of the sun in the sky throughout a day. The programmable stages can comprise a pump such as a hydraulic pump.

In an embodiment, a system further comprises a computer programmable processor in communication with the pump, wherein the computer programmable processor is configured to send a set of instructions to the system. In some instances, the computer programmable processor comprises a network connection configured to receive a set of instructions from an external device. Examples of an external device include, but are not limited to: a server, a personal computer, a cell phone, a personal handheld device, a database, or programmed storage medium.

The computer implemented system can be part of or coupled with a photobioreactor. In some embodiments, the system can be configured or programmed to control and adjust operational parameters of the photobioreactor as well as analyze and calculate values. In some embodiments, the computer implemented system can send and receive control signals to set and control operating parameters of the photobioreactor and, optionally, other related apparatuses. In other embodiments, the computer implemented system can be remotely located with respect to the photobioreactor. It can also be configured to receive data from one or more remote photobioreactors via indirect or direct means, such as through an ethernet connection or wireless connection. The control system can be operated remotely, such as through the Internet.

Part or all of the control of a system or photobioreactor of the invention can be accomplished without a computer (for example, using thermostat to control temperature). Other types of control may be accomplished with physical controls (for example, a diaphragm is passively controlled).

While preferred embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A photobioreactor comprising:
a body having an interior volume for containing a growth medium, wherein the body is configured for the growth of a photosynthetic organism; and
a diaphragm within the body configured to alter the interior volume of the body.

2. The photobioreactor of claim 1, further comprising one or more light sensors positioned on and/or inside the body.

3. The photobioreactor of claim 2, wherein the light sensor is configured to detect optical density of a growth medium.

4. The photobioreactor of claim 1, further comprising a control system for determining a desired interior volume of the body.

5. The photobioreactor of claim 1, wherein the body comprises a surface that is transparent to light.

6. The photobioreactor of claim 5, wherein the surface is configured to allow selected wavelengths of light through the surface and block unselected wavelengths of light.

7. The photobioreactor of claim 6, wherein the unselected wavelengths are in the ultraviolet spectrum.

8. The photobioreactor of claim 1, further comprising a programmable stage configured to tilt the photobioreactor.

9. The photobioreactor of claim 8, wherein the programmable stage comprises one or more diaphragms.

10. The photobioreactor of claim 8, wherein the programmable stage is configured to tilt the photobioreactor based on the position of the sun.

11. The photobioreactor of claim 8, wherein the programmable stage is configured to tilt at least 30 degrees.

12. A system for growing a photosynthetic organism comprising:
one or more photobioreactors,
wherein the photobioreactors include
a body having an adjustable interior volume for containing a growth medium,
wherein the body is configured for the growth of a photosynthetic organism;
a programmable stage configured to tilt the photobioreactor; and
one or more light sensors;
a control system configured to monitor the one or more light sensors and control (a) the adjustable interior volume, (b) the programmable stage, or (c) both (a) and (b).

13. The system of claim 12, wherein the adjustable interior volumes of the photobioreactors are fluidly connected in series.

14. The system of claim 12, wherein the adjustable interior volumes of the photobioreactors are fluidly connected in parallel.

15. A method of producing biomass comprising
growing a photosynthetic organism within a photobioreactor having an adjustable interior volume for containing a growth medium;
evaluating light conditions within the photobioreactor; and
altering the adjustable interior volume based on the light conditions within the photobioreactor.

16. The system of claim 15, wherein the adjustable interior volume of the photobioreactor is altered using a low-pressure hydraulic system to pump a fluid in or out of a diaphragm contained within the photobioreactor.

17. The system of claim 15, further comprising evaluating the position of the sun and orienting the photobioreactor to minimize the angle of incidence for light impinging upon the photobioreactor.

18. The system of claim 17, wherein a low-pressure hydraulic system is utilized for orienting the photobioreactor.
